(19) United States
(12) Patent Application Publication
(10) Pub. No.: US 2026/0325586 A1
(43) Pub. Date: Oct. 22, 2026

(54) PET NUTRITIONAL SUPPLEMENT GRANULE AND PREPARATION METHOD THEREOF

(71) Applicant: Shandong Tu Wang Biotechnology Co., Ltd., Linyi (CN)

(72) Inventors: Jinlong ZHOU, Linyi (CN); Zhida WANG, Beijing (CN); Jinglong WANG, Linyi (CN); Ben LI, Linyi (CN)

(21) Appl. No.: 19/286,237

(22) Filed: Jul. 30, 2025

(30) Foreign Application Priority Data
Apr. 18, 2025 (CN) 202510488934.2

(51) Int. Cl. A61K 35/612; A61K 9/48; A61K 9/50; A61K 31/07; A61K 31/122; A61K 31/195; A61K 31/202; A61K 31/4415; A61K 31/455; A61K 31/51; A61K 31/519; A61K 31/525; A61K 31/593; A61K 31/714; A61K 36/02; A61K 36/42

(52) U.S. Cl. CPC A61K 35/612; A61K 9/4858; A61K 9/4866; A61K 9/5015; A61K 9/5036; A61K 9/5047; A61K 9/5052; A61K 9/5057; A61K 9/5073; A61K 9/5089; A61K 31/07; A61K 31/122; A61K 31/195; A61K 31/202; A61K 31/4415; A61K 31/455; A61K 31/51; A61K 31/519; A61K 31/525; A61K 31/593; A61K 31/714; A61K 36/02; A61K 36/42

(57) ABSTRACT

To solve problems of low bioavailability of Antarctic krill oil and allergic reactions in some pets, a pet nutritional supplement granule and a preparation method thereof are provided. By adding DHA algae oil and pumpkin seed oil, which work synergistically, the bioavailability of the Antarctic krill oil is increased, and allergic symptoms such as diarrhea, skin itching, redness, and ear infections caused by the allergic reactions are effectively alleviated. In addition, pets' aversion to the Antarctic krill oil is reduced through encapsulating the Antarctic krill oil into a nutritional layer. Taste-masking pellets made from strong-tasting vitamin raw materials are incorporated into the nutritional layer to solve a problem of pets' aversion to consuming vitamin supplements. By adjusting the formulation of the taste-masking pellets, pet palatability is improved without the need for an additional flavor layer. Moreover, a controlled-release function is achieved, which alleviates gastrointestinal stress in pets.

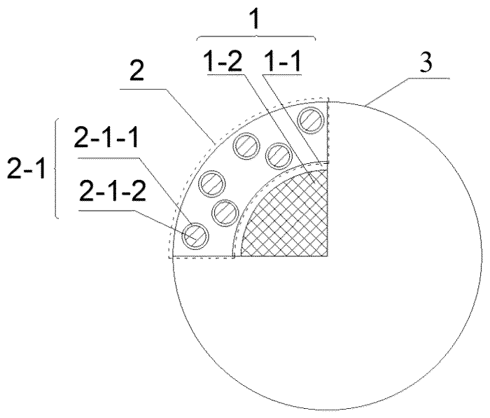

### CROSS-REFERENCE TO RELATED APPLICATION

This application claims priority to Chinese Patent Application No.202510488934.2, filed on Apr. 18, 2025, which is herein incorporated by reference in its entirety.

### TECHNICAL FIELD

The disclosure relates to the technical field of pet foods, and more particularly to a pet nutritional supplement granule and a preparation method thereof.

### BACKGROUND

Antarctic krill oil is a natural lipid extracted from Antarctic krill, rich in various nutrients that are beneficial to the health of pets. The Antarctic krill oil is particularly rich in ω-3 fatty acids, especially eicosapentaenoic acid (EPA) and docosahexaenoic acid (DHA), which possess strong anti-inflammatory properties that can effectively reduce joint inflammation and alleviate pain and swelling caused by arthritis and other diseases. In addition, the ω-3 fatty acids can also support joint lubrication, helping to effectively prevent the occurrence ofjoint diseases in pets that are highly active or aging. However, existing Antarctic krill oil supplements for the pets typically come in the form of soft capsules, capsules, or drops, which have poor palatability for the pets and also have low bioavailability of the Antarctic krill oil. In addition, some pets may experience allergic reactions such as soft stools, skin itching, redness, and ear infections after consuming these supplements.

Moreover, the above Antarctic krill oil products have a relatively limited nutritional profile. If there is a need to supplement other types of nutrients, such as calcium, iron, zinc, and other nutritional elements, separate supplementation is required.

Therefore, how to provide a palatable supplement that can deliver the Antarctic krill oil along with other nutritional components, enhance the bioavailability of the Antarctic krill oil, improve pet palatability, and alleviate the allergic reactions, has become an urgent problem to be solved.

### SUMMARY

In order to solve the above technical problems, the disclosure provides a pet nutritional supplement granule and a preparation method thereof.

In a first aspect, the disclosure provides a pet nutritional supplement granule, including a burst bead core material and a nutritional layer covering a surface of the burst bead core material. The burst bead core material includes a wall material and a core liquid encapsulated within the wall material. In terms of parts by weight, the core liquid includes 75 parts to 85 parts of Antarctic krill oil, 15 parts to 25 parts of DHA algal oil, and 5 parts to 10 parts of pumpkin seed oil. The nutritional layer includes taste-masking pellets, and each of the taste-masking pellets includes a pellet core and a coating layer covering a surface of the pellet core. The core pellet contains vitamins.

The coating layer includes a first animal-derived meat, an amino acid, salt, sodium alginate, polyethylene glycol, hydroxypropyl methylcellulose, and zein.

For example, the parts by weight of the Antarctic krill oil may be 75 parts, 77 parts, 80 parts, 82 parts, or 85 parts; the parts by weight of the DHA algal oil may be 15 parts, 17 parts, 20 parts, 23 parts, or 25 parts; and the parts by weight of the pumpkin seed oil may be 5 parts, 6 parts, 7 parts, 8 parts, 9 parts, or 10 parts. However, the disclosure is not limited to the listed values, and other values not listed within the above ranges are also applicable.

On one hand, the disclosure solves existing problems in the related art of low bioavailability of the Antarctic krill oil and allergic reactions in some pets. By incorporating the DHA algal oil and the pumpkin seed oil, which work synergistically, the bioavailability of the Antarctic krill oil is increased, and allergic symptoms such as diarrhea, skin itching, erythema, and ear infections caused by the allergic reactions are effectively alleviated. In addition, the encapsulation of the Antarctic krill oil with the nutritional layer reduces pets' aversion to the Antarctic krill oil. On other hand, the disclosure also solves a problem of pets' aversion to consuming vitamin supplements by incorporating the taste-masking pellets made from strong-tasting vitamin raw materials into the nutritional layer. By adjusting the formulation of the taste-masking pellets, pet palatability is improved without the need for the additional flavor layer. moreover, a controlled-release function is achieved, which alleviates gastrointestinal stress in pets.

In the disclosure, an amount of the pumpkin seed oil added should not be excessive. An excessive amount of the pumpkin seed oil not only has limited effect on improving the bioavailability of the Antarctic krill oil but also increases the risk of allergic reactions in the pets.

The following are specific technical solutions in the disclosure, but are not intended to limit the technical solutions provided in the disclosure. Through the following technical solutions, technical purposes and beneficial effects of the disclosure can be better achieved and realized.

In a specific technical solution of the disclosure, in the burst bead core material, a mass ratio of the wall material to the core liquid is (0.3-0.4): 1.

In a specific technical solution of the disclosure, a diameter of the burst bead core material is in a range of 5 millimeters (mm) to 6 mm, such as 5 mm, 5.2 mm, 5.4 mm, 5.6 mm 5.8 mm, or 6 mm, but is not limited to the listed values, and other values not listed within this range are also applicable.

The wall material includes glycerin, sorbitol, and an edible gum.

In an embodiment, a mass ratio of the glycerin, the sorbitol, and the edible gum is (80-100):(20-40):(2800-3200), such as 90:30:300, 80:20:2800, 100:40:3200, or 95:35:2900, but is not limited to the listed values, and other values not listed within this range are also applicable.

In an embodiment, the wall material further includes a coloring agent. The coloring agent is a food-grade pigment, which may be added as needed.

In an embodiment, the edible gum includes gelatin.

In a specific technical solution of the disclosure, the vitamins include one or more selected from the group consisting of vitamin B1, vitamin B2, vitamin B6, vitamin B12, calcium D-pantothenate, and niacin.

In an embodiment, the vitamins further include one or more selected from the group consisting of vitamin A, vitamin D3, vitamin K, and folic acid.

In the disclosure, types of the vitamins are not limited to the above substrates, other vitamins that are beneficial to the pets may also be added. Moreover, an amount of the vitamin added can also be adjusted according to product requirements. Further details are not repeated here.

In an embodiment, the nutritional layer further includes a first excipient, and the first excipient includes one or more selected from the group consisting of starch, maltodextrin, and white sugar.

In a specific technical solution of the disclosure, the first animal-derived meat includes one or more selected from the group consisting of chicken meat, chicken liver, and beef.

In an embodiment, the amino acid includes one or more selected from the group consisting of taurine, proline, lysine, glycine, and glutamic acid.

In a specific technical solution of the disclosure, in terms of mass percent, the coating layer includes 60 wt % to 75 wt % of the first animal-derived meat, 10 wt % to 20 wt % of the amino acid, 1 wt % to 3 wt % of the salt, 5 wt % to 15 wt % of the sodium alginate, 1 wt % to 5 wt % of the polyethylene glycol, 3 wt % to 7 wt % of the hydroxypropyl methylcellulose, and 5 wt % to 15 wt % of the zein. For example, a content of the first animal-derived meat can be 60 wt %, 65 wt %, 70 wt % or 75 wt %, etc.; a content of the amino acid can be 10 wt %, 12 wt %, 14 wt %, 16 wt %, 18 wt %, or 20 wt %, etc.; a content of the salt can be 1 wt %, 2 wt %, or 3 wt %, etc.; a content of the sodium alginate can be 5 wt %, 7 wt %, 10 wt %, 13 wt %, or 15 wt %, etc.; a content of the polyethylene glycol can be 1 wt %, 2 wt %, 3 wt %, 4 wt %, or 5 wt %, etc.; a content of the hydroxypropyl methylcellulose can be 3 wt %, 4 wt %, 5 wt %, 6 wt %, or 7 wt %, etc.; a content of the zein can be 5 wt %, 10 wt %, 12 wt %, or 15 wt %, etc. However, the disclosure is not limited to the listed values, and other values not listed within the above ranges are also applicable.

In a specific technical solution of the disclosure, a diameter of each of the taste-masking pellets is in a range of 0.9 mm to 1.1 mm, such as 0.9 mm, 1.0 mm, or 1.1 mm, but is not limited to the listed values, and other values not listed within the above range are also applicable.

In an embodiment, a thickness of the nutritional layer is in a range of 2 mm to 3 mm, such as 2 mm, 2.2 mm, 2.4 mm, 2.6 mm, 2.8 mm or 3 mm, but is not limited to the listed values, and other values not listed within the above range are also applicable.

In a specific technical solution of the disclosure, the pet nutritional supplement granule further includes a flavor layer covering a surface of the nutritional layer.

In an embodiment, the flavor layer includes a second animal-derived meat and a second excipient.

In an embodiment, the second animal-derived meat includes one or more selected from the group consisting of chicken meat, beef, and chicken liver.

In an embodiment, the second excipient includes at least one of starch and maltodextrin.

In an embodiment, a thickness of the flavor layer is in a range of 2 mm to 3 mm, such as 2 mm, 2.2 mm, 2.4 mm, 2.6 mm, 2.8 mm or 3 mm, but is not limited to the listed values, and other values not listed within the above range are also applicable.

In a second aspect, the disclosure provides a preparation method of the pet nutritional supplement granule as described in the first aspect, including:
step (1), preparing the core liquid and a wall material adhesive solution, making the core liquid and the wall material adhesive solution to form a concentric droplet by using a microencapsulation technology, dropping the concentric droplet into a coagulation liquid to form a wet bead, and drying the wet bead to obtain the burst bead core material;
step (2), preparing powdered vitamins, making the powdered vitamins to form the pellet cores by using a centrifugal granulation method, coating the pellet cores to obtain the taste-masking pellets; and
step (3), preparing powder for the nutritional layer, making the powder to form the nutritional layer on the surface of the burst bead core material obtained in step (1) by using the centrifugal granulation method, followed by drying, thereby obtaining the pet nutritional supplement granule.

The powder for the nutritional layer includes the taste-masking pellets.

Steps (1) and (2) are not necessarily performed in sequence.

The product obtained by the preparation method described in the disclosure has a stable structure and good powder coating properties. The production process is simple and is conducive to industrial application.

In a specific technical solution of the disclosure, the preparation method further includes: after the drying in step (3), using the centrifugal granulation method to forming a flavor layer on a surface of the nutritional layer by using the centrifugal granulation method, followed by drying to obtain the pet nutritional supplement granule.

In a specific technical solution of the disclosure, a temperature of the drying in step (1) is in a range of 25° C. to 35° C., such as 25° C., 30° C., or 35° C., but is not limited to the listed values, and other values not listed within the above range are also applicable.

In an embodiment, a moisture content of the burst bead core material obtained after the drying in step (1) is less than 8 wt %, such as 2 wt %, 4 wt %, 6 wt %, or 8 wt %, but is not limited to the listed values, and other values not listed within the above range are also applicable.

In an embodiment, a temperature of the drying in step (3) is in a range of 60° C. to 80° C., such as 60° C., 65° C., 70° C., 75° C., or 80° C., but is not limited to the listed values, and other values not listed within the above range are also applicable.

In an embodiment, a moisture content of the pet nutritional supplement granule obtained after the drying in step (3) is less than 8 wt %, such as 2 wt %, 4 wt %, 6 wt %, or 8 wt %, but is not limited to the listed values, and other values not listed within the above range are also applicable.

In a specific technical solution of the disclosure, specific operations of step (1) include:
S11, preparation of the core liquid, including: mixing the 75 parts to 85 parts of Antarctic krill oil, the 15 parts to 25 parts of DHA algal oil, and the 5 parts to 10 parts of pumpkin seed oil at a temperature in a range of 15° C. to 35° C.;
S12, preparation of the wall material adhesive solution, including: mixing glycerin, sorbitol, and an edible gum in a mass ratio of (80-100):(20-40):(2800-3200) with water at 50-70° C., followed by stirring uniformly to obtain a mixed solution, and heating the mixed solution to 80° C. to 85° C. for 1.5 hours to 2.5 hours; and
S13, adding the core liquid and the adhesive solution respectively to an oil pot and a glue pot of a dropping pill machine, setting a dripping speed of a dropper of the dropping pill machine to 13 drops per second (dps), and dropping the concentric droplet into the coagulation liquid to form the wet bead; and drying the wet bead at a temperature in a range of 25° C. to 35° C., and removing the coagulation liquid from a surface of the wet bead with absorbent paper until a moisture content of the wet bead is less than 8 wt %, thereby obtaining the burst bead core material.

A temperature of the oil pot is set to 15° C. to 30° C., and a temperature of the glue pot is set to 70° C. to 90° C.

In a specific technical solution of the disclosure, specific operations of step (2) include:
S21, preparation of the pellet cores, including: placing the powdered vitamins into a centrifugal granulation machine for rounding, followed by drying to obtain the pellet cores with a moisture content in a range of 8 wt % to 10 wt %;
S22, preparation of a coating solution, including: dissolving 60 wt % to 75 wt % of the first animal-derived meat, 10 wt % to 20 wt % of the amino acid, 1 wt % to 3 wt % of the salt, 5 wt % to 15 wt % of the sodium alginate, 1 wt % to 5 wt % of the polyethylene glycol, 3 wt % to 7 wt % of the hydroxypropyl methylcellulose, and 5 wt % to 15 wt % of the zein into water to obtain the coating solution; and
S23, placing the pellet cores in a bottom-spray fluidized bed coating machine, and coating the coating solution on the pellet cores by activating a bottom spray nebulizer of the bottom-spray fluidized bed coating machine to obtain the taste-masking pellets with a set diameter.

In the disclosure, an amount of the water added to the coating solution can be determined by those skilled in the art based on experience, as long as it reaches a coating state.

In a specific technical solution of the disclosure, specific operations of step (3) include:
S31, preparation of the powder for the nutritional layer, including: mixing the taste-masking pellets obtained in step (2) with a first excipient to obtain the powder for the nutritional layer; and
S32, placing the burst bead core material obtained in step (1) in a centrifugal granulation machine, setting an operating frequency of the centrifugal granulation machine to 35 Hertz (Hz) to 45 Hz, and spraying water to moisten the surface of the burst bead core material, followed by performing a rolling operation by spraying the powder for the nutritional layer to obtain a moisturizing pet nutritional supplement granule; and taking out the moisturizing pet nutritional supplement granule when a diameter of the moisturizing pet nutritional supplement granule reaches a set diameter, and drying the moisturizing pet nutritional supplement granule at 60° C. to 80° C. until a moisture content of the moisturizing pet nutritional supplement granule is less than 8 wt %, thereby obtaining the pet nutritional supplement granule.

In a specific technical solution of the disclosure, in each step of the preparation process, standard-sized products are screened out using a standard sieve.

In a specific technical solution of the disclosure, in the process of preparing the burst bead core materials, after screening out the burst bead core materials that meet the standard size using the standard sieve, irregularly shaped, leaking, or damaged substandard burst bead core materials are further sorted out manually.

Compared to the related art, technical solutions provided by embodiments of the disclosure may be achieve the following advantages.

(1) The disclosure solves existing problems in the related art of low bioavailability of the Antarctic krill oil and allergic reactions in some pets. By adding the DHA algae oil and the pumpkin seed oil, which work synergistically, the bioavailability of the Antarctic krill oil is increased, and allergic symptoms such as diarrhea, skin itching, erythema, and ear infections caused by the allergic reactions are effectively alleviated. In addition, pets' aversion to the Antarctic krill oil can be reduced by encapsulating the Antarctic krill oil into the nutritional layer.

(2) The disclosure also solves a problem of pets' aversion to consuming vitamin supplements by incorporating the taste-masking pellets made from strong-tasting vitamin raw materials into the nutritional layer. By adjusting the formulation of the taste-masking pellets, pet palatability is improved without the need for the additional flavor layer. Moreover, a controlled-release function is achieved, which alleviates gastrointestinal stress in the pets.

(3) The product prepared according to the disclosure has a stable structure and good powder coating properties. The production process is simple and is conducive to industrial application.

### BRIEF DESCRIPTION OF DRAWING

Attached drawing, which is incorporated into and constitute a part of the specification, illustrate embodiments in accordance with the disclosure and together with the specification explain the principles of the disclosure.

In order to more clearly illustrate technical solutions in embodiments of the disclosure or in the related art, a brief description to the attached drawing necessary for describing the embodiments or the related art will now be given. It is apparent that for those skilled in the art, other attached drawing can be obtained based on the attached drawing without involving any inventive step.

FIGURE illustrates a cross-sectional view of a pet nutritional supplement granule according to a first embodiment of the disclosure.

### DESCRIPTION OF REFERENCE SIGNS

1. burst bead core material; 1-1. wall material; 1-2. core liquid; 2. nutritional layer; 2-1. taste-masking pellet; 2-1-1. coating layer; 2-1-2. pellet core; 3. flavor layer.

### DETAILED DESCRIPTION OF EMBODIMENTS

To provide a clearer understanding of the above purposes, features, and advantages of the disclosure, solutions of the disclosure will be further described below. It should be noted that, where there is no conflict, embodiments disclosed herein and features thereof can be combined with each other.

In following description, numerous specific details are set forth to provide a thorough understanding of the disclosure. However, the disclosure may be implemented in other ways different from those described herein. It is apparent that the embodiments described in the specification are only a part of the embodiments of the disclosure, and not all of them.

### First Embodiment

The first embodiment provides a pet nutritional supplement granule, and a cross-sectional view of the pet nutritional supplement granule is shown in FIGURE. The pet nutritional supplement granule includes a burst bead core material 1 with a diameter of 5 mm and a nutritional layer 2 with a thickness of 3 mm, and a surface of the burst bead core material 1 is covered with the nutritional layer 2.

The burst bead core material 1 includes a wall material 1-1 and a core liquid 1-2 encapsulated within the wall material 1-1.

In terms of parts by weight, the core liquid 1-2 includes 85 parts of Antarctic krill oil, 20 parts of DHA algal oil, and 5 parts of pumpkin seed oil.

The nutritional layer 2 includes taste-masking pellets 2-1 with a diameter of 1 mm, starch, maltodextrin, and white sugar.

Each of the taste-masking pellets 2-1 includes a pellet core 2-1-2 and a coating layer 2-1-1 covering a surface of the pellet core 2-1-2.

The pellet core 2-1-2 contains vitamins.

The coating layer 2-1-1 include 25 wt % of chicken meat, 20 wt % of chicken liver, 20 wt % of beef, 3 wt % of taurine, 1 wt % of proline, 2 wt % of lysine, 1 wt % of glycine, 3 wt % of glutamic acid, 1 wt % of salt, 5 wt % of sodium alginate, 1 wt % of polyethylene glycol, 5 wt % of hydroxypropyl methylcellulose, and 13 wt % of zein.

A preparation method of the pet nutritional supplement granule includes the following steps.

### (1) Preparation of the Burst Bead Core Material 1

S1, preparation of the core liquid 1-2:20 kilograms (kg) of the Antarctic krill oil, 5 kg of the DHA algal oil, and 1.25 kg of the pumpkin seed oil are mixed at 25° C. to obtain the core liquid 1-2.

S2, preparation of an adhesive solution (raw material of the wall material 1-1):at 60° C., 90 grams (g) of glycerol, 30 g of sorbitol solution, and 3 kg of gelatin are added sequentially to 20 kg of purified water, followed by stirring uniformly to obtain a mixed solution. Then the mixed solution is heated to 80° C. for 2 hours to obtain the adhesive solution.

S3, the core liquid 1-2 is added to an oil pot of a dropping pill machine, and the adhesive solution is added to a glue pot of the dropping pill machine. After the adhesive solution is allowed to stand for 2 hours to completely remove bubbles, a dripping process is performed. A dripping speed of a dropper of the dropping pill machine is set to 13 dps. Generated concentric droplets (i.e., the adhesive solution encapsulating the core liquid 1-2) are dropped into liquid paraffin oil to form wet beads. The wet beads are then placed into a rotating cage drying machine for drying. Simultaneously, absorbent paper is used to remove the paraffin oil from surfaces of the wet beads until a moisture content of each of the wet beads is less than 8 wt %, thereby obtaining the burst bead core materials 1.

The burst bead core materials 1 are screened using a standard sieve to select products (i.e., the burst bead core materials 1) with a diameter of 5 mm. Then irregularly shaped, leaking, or damaged substandard products are sorted out manually. An output of the burst bead core materials 1 is 26 kg.

### (2) Coating of the Nutritional Layer 2

S4, preparation of the pellet cores 2-1-2:1.5 g of powdered vitamin A, 5 g of powdered vitamin D3, 1 g of powdered vitamin B1, 2 g of powdered vitamin B2, 1 g of powdered vitamin B6, 0.02 g of powdered vitamin B12, 10 g of powdered niacin, 0.2 g of powdered folic acid, and 10 g of powdered calcium D-pantothenate are placed into a centrifugal granulation machine for rounding, followed by drying to obtain the pellet cores with a moisture content of 9 wt %.

S5, preparation of a coating solution: 2.5 kg of chicken meat powder, 2 kg of chicken liver powder, 2 kg of beef powder, 0.3 kg of the taurine, 0.1 kg of the proline, 0.2 kg of the lysine, 0.1 kg of the glycine, 0.3 kg of the glutamic acid, 0.1 kg of the salt, 0.5 kg of the sodium alginate, 0.1 kg of polyethylene glycol 400, 0.5 kg of the hydroxypropyl methylcellulose, and 1.3 kg of the zein are dissolved into 56 kg of water to obtain the coating solution.

S6, preparation of the taste-masking pellets 2-1:100 kg of the pellet cores 2-1-2 are placed in a bottom-spray fluidized bed coating machine. A bottom spray nebulizer of the bottom-spray fluidized bed coating machine is activated to coat the coating solution on the pellet cores 2-1-2 to obtain the taste-masking pellets 2-1 with a diameter of 1 mm.

S7, preparation of mixed powder (raw material for the nutritional layer 2): 50 kg of the maltodextrin, 30 kg of pregelatinized corn starch, and 20 kg of the taste-masking pellets 2-1 are mixed to obtain the mixed powder.

S8, the qualified burst bead core materials 1 obtained in step (1) are placed in the centrifugal granulation machine. An operating frequency of the centrifugal granulation machine is set to 40 Hz. The surfaces of the burst bead core materials 1 are moistened with water, and the mixed powder is sprayed for rolling to obtain moisturizing pet nutritional supplement granules. The moisturizing pet nutritional supplement granules are taken out when they have a diameter of 8 mm. The moisturizing pet nutritional supplement granules are dried at 70° C. until they have a moisture content less than 8 wt %, thereby obtaining the pet nutritional supplement granules.

### Second Embodiment

The second embodiment provides a pet nutritional supplement granule and a preparation method thereof. The pet nutritional supplement granule in the second embodiment is the same as that described in the first embodiment, except that the core liquid 1-2 includes 18.75 kg of Antarctic krill oil, 6.25 kg of DHA algal oil, and 2.5 kg of pumpkin seed oil.

A preparation method of the pet nutritional supplement granule in the second embodiment is the same as that described in the first embodiment, except that in S1, the 18.75 kg of Antarctic krill oil, the 6.25 kg of DHA algal oil, and the 2.5 kg of pumpkin seed oil are mixed at 25° C. to obtain the core liquid 1-2.

In this preparation process, an output of the burst bead core materials 1 is 24.5 kg.

### Third Embodiment

The third embodiment provides a pet nutritional supplement granule and a preparation method thereof. The pet nutritional supplement granule in the third embodiment is the same as that described in the first embodiment, except that the core liquid 1-2 includes 21.25 kg of Antarctic krill oil, 3.75 kg of DHA algal oil, and 2 kg of pumpkin seed oil.

A preparation method of the pet nutritional supplement granule in the third embodiment is the same as that described in the first embodiment, except that in S1, the 21.25 kg of Antarctic krill oil, the 3.75 kg of DHA algal oil, and the 2 kg of pumpkin seed oil are mixed at 25° C. to obtain the core liquid 1-2.

In this preparation process, an output of the burst bead core materials 1 is 22 kg.

### First Comparative Embodiment

The first comparative embodiment provides a pet nutritional supplement granule and a preparation method thereof. The pet nutritional supplement granule in the first comparative embodiment is the same as that described in the first embodiment, except that the core liquid 1-2 includes only 26.25 kg of Antarctic krill oil.

A preparation method of the pet nutritional supplement granule in the first comparative embodiment is the same as that described in the first embodiment, except that in S1, the core liquid 1-2 includes only the 26.25 kg of Antarctic krill oil at 25° C.

In this preparation process, an output of the burst bead core materials 1 is 15 kg.

### Second Comparative Embodiment

The second comparative embodiment provides a pet nutritional supplement granule and a preparation method thereof. The pet nutritional supplement granule in the second comparative embodiment is the same as that described in the first embodiment, except that the core liquid 1-2 includes 21 kg of Antarctic krill oil and 5.25 kg of DHA algal oil.

Under a condition of ensuring a total mass of the core liquid remains unchanged, a reduced weight of pumpkin seed oil is proportionally added to the Antarctic krill oil and the DHA algal oil.

A preparation method of the pet nutritional supplement granule in the second comparative embodiment is the same as that described in the first embodiment, except that in S1, the 21 kg of Antarctic krill oil and the 5.25 kg of DHA algal oil are mixed at 25° C. to obtain the core liquid 1-2.

In this preparation process, an output of the burst bead core materials 1 is 15.4 kg.

### Third Comparative Embodiment

The third comparative embodiment provides a pet nutritional supplement granule and a preparation method thereof. The pet nutritional supplement granule in the third comparative embodiment is the same as that described in the first embodiment, except that the core liquid 1-2 includes 24.71 kg of Antarctic krill oil and 1.54 kg of pumpkin seed oil.

Under a condition of ensuring a total mass of the core liquid remains unchanged, a reduced weight of DHA algal oil is proportionally added to the Antarctic krill oil and the pumpkin seed oil.

A preparation method of the pet nutritional supplement granule in the third comparative embodiment is the same as that described in the first embodiment, except that in S1, the 24.71 kg of Antarctic krill oil and the 1.54 kg of pumpkin seed oil are mixed at 25C.° to obtain the core liquid 1-2.

In this preparation process, an output of the burst bead core materials 1 is 15.6 kg.

### Fourth Comparative Embodiment

The fourth comparative embodiment provides a pet nutritional supplement granule and a preparation method thereof. The pet nutritional supplement granule in the fourth comparative embodiment is the same as that described in the first embodiment, except that the core liquid 1-2 includes 18.38 kg of Antarctic krill oil, 6.12 kg of DHA algal oil and 3 kg of pumpkin seed oil.

Under a condition of ensuring a total mass of the core liquid remains unchanged, an increased weight of the pumpkin seed oil is offset by proportional reductions in the Antarctic krill oil and the DHA algal oil.

A preparation method of the pet nutritional supplement granule in the fourth comparative embodiment is the same as that described in the first embodiment, except that in S1, the 18.38 kg of Antarctic krill oil, the 6.12 kg of DHA algal oil and the 3 kg of pumpkin seed oil are mixed at 25° C. to obtain the core liquid 1-2.

In this preparation process, an output ofthe burst bead core materials 1 is 15 kg.

### Product Performance Testing

The bioavailability of the Antarctic krill oil in the pet nutritional supplement granules obtained from the first embodiment to the third embodiment and the first comparative embodiment to the fourth comparative embodiment are measured.

The bioavailability of the Antarctic krill oil is calculated as a total mass of the burst bead core materials divided by a sum of a total mass of the core liquid and a total mass of the edible gum, multiplied by 10000, where the edible gum is the gelatin, and amounts of the glycerin and the sorbitol are not included in the calculation.

A test result of the bioavailability of the Antarctic krill oil in the pet nutritional supplement granules obtained from the first embodiment to the third embodiment and the first comparative embodiment to the fourth comparative embodiment is shown in Table 1.

TABLE 1

| | total output | total mass | total mass | bioavail- |
|---|---|---|---|---|
| | mass of burst | of core | of edible | ability of |
| | bead core | liquid | gum | Antarctic |
| | material/kg | used/kg | used/kg | krill oil |
| first | 26.0 | 26.25 | 3 | 88.89% |
| embodiment | | | | |
| second | 24.5 | 27.50 | 3 | 80.33% |
| embodiment | | | | |
| third | 22.0 | 27.00 | 3 | 73.33% |
| embodiment | | | | |
| first | 15.0 | 26.25 | 3 | 51.28% |
| comparative | | | | |
| embodiment | | | | |
| second | 15.4 | 26.25 | 3 | 52.60% |
| comparative | | | | |
| embodiment | | | | |
| third | 15.6 | 26.25 | 3 | 53.33% |
| comparative | | | | |
| embodiment | | | | |
| fourth | 15.0 | 27.50 | 3 | 49.18% |
| comparative | | | | |
| embodiment | | | | |

The following conclusions can be obtained from Table 1.

The test result shows that the pet nutritional supplement granule of the disclosure ensures the structural stability of Antarctic krill oil burst beads by adding the DHA algal oil and the pumpkin seed oil, thereby effectively increasing the bioavailability of the Antarctic krill oil from 49% to over 73%.

The core liquid of the burst bead core material in the first comparative embodiment only includes the Antarctic krill oil, which results in a lower bioavailability.

In the second and third comparative embodiments, only one of the DHA algal oil or the pumpkin seed oil is added. Without the synergistic effect of both, the improvement in the bioavailability of the Antarctic krill oil is limited.

In the fourth comparative embodiment, the addition of the pumpkin seed oil is excessive, which not only reduced the bioavailability of the Antarctic krill oil but also increased the risk of allergic reactions in pets.

It should be noted that, in the specification, relationship terms such as “first” and “second,” are used solely to distinguish one entity or operation from another entity or operation, and do not necessarily require or imply any actual relationship or sequence between these entities or operations. Moreover, terms “include”, “contain” or any other variants are intended to cover non-exclusive inclusion. As such, a process, a method, an article, or an apparatus that includes a series of elements includes not only those elements but also other elements not explicitly listed, or elements inherent to such process, method, article, or apparatus. Unless otherwise limited, an element defined by a phrase “including one . . . ” does not exclude the presence of additional identical elements in the process, the method, the article, or the apparatus that includes the elements.

The embodiments described above are merely specific implementations of the disclosure, enabling those skilled in the art to understand or practice the disclosure. Various modifications to these embodiments will be apparent to those skilled in the art, and the general principles defined herein may be implemented in other embodiments without departing from the spirit or scope of the disclosure. Therefore, the disclosure is not limited to the embodiments described herein but should be accorded the broadest scope consistent with the principles and novel features disclosed herein.

What is claimed is:

1. A pet nutritional supplement granule, comprising a burst bead core material and a nutritional layer covering a surface of the burst bead core material;
wherein the burst bead core material comprises a wall material and a core liquid encapsulated within the wall material;
wherein in terms of parts by weight, the core liquid comprises 75 parts to 85 parts of Antarctic krill oil, 15 parts to 25 parts of docosahexaenoic acid (DHA) algal oil, and 5 parts to 10 parts of pumpkin seed oil;
wherein the nutritional layer comprises taste-masking pellets, and each of the taste-masking pellets comprises a pellet core and a coating layer covering a surface of the pellet core;
wherein the pellet core contains vitamins; and
wherein the coating layer comprises an animal-derived meat, an amino acid, salt, sodium alginate, polyethylene glycol, hydroxypropyl methylcellulose, and zein.

2. The pet nutritional supplement granule as claimed in claim 1, wherein a diameter of the burst bead core material is in a range of 5 millimeters (mm) to 6 mm;
wherein the wall material comprises glycerin, sorbitol, and an edible gum;
wherein a mass ratio of the glycerin, the sorbitol, and the edible gum is (80-100) (20-40):(2800-3200);
wherein the wall material further comprises a coloring agent; and
wherein the edible gum comprises gelatin.

3. The pet nutritional supplement granule as claimed in claim 2, wherein the vitamins comprise one or more selected from the group consisting of vitamin B1, vitamin B2, vitamin B6, vitamin B12, calcium D-pantothenate, and niacin;
wherein the vitamins further comprise one or more selected from the group consisting of vitamin A, vitamin D3, vitamin K, and folic acid; and
wherein the nutritional layer further comprises an excipient, and the excipient comprises one or more selected from the group consisting of starch, maltodextrin, and white sugar.

4. The pet nutritional supplement granule as claimed in claim 1, wherein the animal-derived meat comprises one or more selected from the group consisting of chicken meat, chicken liver, and beef; and
wherein the amino acid comprises one or more selected from the group consisting of taurine, proline, lysine, glycine, and glutamic acid.

5. The pet nutritional supplement granule as claimed in claim 4, wherein in terms of mass percent, the coating layer comprises 60 wt % to 75 wt % of the animal-derived meat, 10 wt % to 20 wt % of the amino acid, 1 wt % to 3 wt % of the salt, 5 wt % to 15 wt % of the sodium alginate, 1 wt % to 5 wt % of the polyethylene glycol, 3 wt % to 7 wt % of the hydroxypropyl methylcellulose, and 5 wt % to 15 wt % of the zein.

6. The pet nutritional supplement granule as claimed in claim 1, wherein a diameter of each of the taste-masking pellets is in a range of 0.9 mm to 1.1 mm; and a thickness of the nutritional layer is in a range of 2 mm to 3 mm.

7. A preparation method of the pet nutritional supplement granule as claimed in claim 1, comprising:
step (1), preparing the core liquid and a wall material adhesive solution, making the core liquid and the wall material adhesive solution to form a concentric droplet by using a microencapsulation technology, dropping the concentric droplet into a coagulation liquid to form a wet bead, and drying the wet bead to obtain the burst bead core material;
step (2), preparing powdered vitamins, making the powdered vitamins to form the pellet cores by using a centrifugal granulation method, and coating the pellet cores to obtain the taste-masking pellets; and
step (3), preparing powder for the nutritional layer, making the powder to form the nutritional layer on the surface of the burst bead core material obtained in step (1) by using the centrifugal granulation method, followed by drying, thereby obtaining the pet nutritional supplement granule; and
wherein the powder for the nutritional layer comprises the taste-masking pellets.

8. The preparation method as claimed in claim 7, wherein the preparation method further comprises: after the drying in step (3), forming a flavor layer on a surface of the nutritional layer by using the centrifugal granulation method, followed by drying to obtain the pet nutritional supplement granule.

9. The preparation method as claimed in claim 8, wherein a temperature of the drying in step (1) is in a range of 25° C. to 35° C., and a moisture content of the burst bead core material obtained after the drying in step (1) is less than 8 wt %.

10. The preparation method as claimed in claim 7, wherein a temperature of the drying in step (3) is in a range of 60° C. to 80° C., and a moisture content of the pet nutritional supplement granule obtained after the drying in step (3) is less than 8 wt %.

11. The preparation method as claimed in claim 7, wherein step (1) comprises:
S11, preparation of the core liquid, comprising: mixing the 75 parts to 85 parts of Antarctic krill oil, the 15 parts to 25 parts of DHA algal oil, and the 5 parts to 10 parts of pumpkin seed oil at a temperature in a range of 15° C. to 35° C.;
S12, preparation of the wall material adhesive solution, comprising: mixing glycerin, sorbitol, and an edible gum in a mass ratio of (80-100):(20-40):(2800-3200) with water at 50° C. to 70° C., followed by stirring uniformly to obtain a mixed solution, and heating the mixed solution to 80° C. to 85° C. for 1.5 hours to 2.5 hours; and
S13, adding the core liquid and the adhesive solution respectively to an oil pot and a glue pot of a dropping pill machine, setting a dripping speed of a dropper of the dropping pill machine to 13 drops per second (dps), and dropping the concentric droplet into the coagulation liquid to form the wet bead; and drying the wet bead at a temperature in a range of 25° C. to 35° C., and removing the coagulation liquid from a surface of the wet bead with absorbent paper until a moisture content of the wet bead is less than 8 wt %, thereby obtaining the burst bead core material.

12. The preparation method as claimed in claim 7, wherein step (2) comprises:
S21, preparation of the pellet cores, comprising: placing the powdered vitamins into a centrifugal granulation machine for rounding, followed by drying to obtain the pellet cores with a moisture content in a range of 8 wt % to 10 wt %;
S22, preparation of a coating solution, comprising: dissolving 60 wt % to 75 wt % of the animal-derived meat, 10 wt % to 20 wt % of the amino acid, 1 wt % to 3 wt % of the salt, 5 wt % to 15 wt % of the sodium alginate, 1 wt % to 5 wt % of the polyethylene glycol, 3 wt % to 7 wt % of the hydroxypropyl methylcellulose, and 5 wt % to 15 wt % of the zein into water to obtain the coating solution; and
S23, placing the pellet cores in a bottom-spray fluidized bed coating machine, and coating the coating solution on the pellet cores by activating a bottom spray nebulizer of the bottom-spray fluidized bed coating machine to obtain the taste-masking pellets with a set diameter.

13. The preparation method as claimed in claim 7, wherein step (3) comprises:
S31, preparation of the powder for the nutritional layer, comprising: mixing the taste-masking pellets obtained in step (2) with an excipient to obtain the powder for the nutritional layer; and
S32, placing the burst bead core material obtained in step (1) in a centrifugal granulation machine, setting an operating frequency of the centrifugal granulation machine to 35 Hertz (Hz) to 45 Hz, and spraying water to moisten the surface of the burst bead core material, followed by performing a rolling operation by spraying the powder for the nutritional layer to obtain a moisturizing pet nutritional supplement granule; and taking out the moisturizing pet nutritional supplement granule when a diameter of the moisturizing pet nutritional supplement granule reaches a set diameter, and drying the moisturizing pet nutritional supplement granule at 60° C. to 80° C. until a moisture content of the moisturizing pet nutritional supplement granule is less than 8 wt %, thereby obtaining the pet nutritional supplement granule.
